(19) United States
(12) Patent Application Publication
(10) Pub. No.: US 2026/0331234 A1
(43) Pub. Date: Oct. 22, 2026

(54) NUTRITIONAL AND HEALTH STATUS ANALYSIS SYSTEM USING URINE TEST KIT

(71) Applicant: Yuurea Inc., Aichi (JP)

(72) Inventors: Shogo MIZUNO, Aichi (JP); Kenji Saito, Aichi (JP); Shintaro Oyama, Aichi (JP)

(21) Appl. No.: 19/565,419

(22) Filed: Mar. 12, 2026

Related U.S. Application Data
(63) Continuation of application No. PCT/JP2024/030830, filed on Aug. 28, 2024.

(30) Foreign Application Priority Data
Sep. 14, 2023 (JP) 2023-149431

(51) Int. Cl. G16H 10/40; G16H 10/20; G16H 10/60

(52) U.S. Cl. CPC G16H 10/40; G16H 10/20; G16H 10/60

(57) ABSTRACT

A client terminal in a nutritional and health status analysis system includes: a urine test kit color data input unit for inputting color data of a urine test kit immersed in urine collected by a user; a question data input unit for inputting question data regarding the user's physical condition within one week from urine collection; a urine test kit color data analysis unit; a question data analysis unit; and a nutritional and health status evaluation unit that evaluates, by machine learning, the user's nutritional and health status based on basic information including sex and age, registration information including lifestyle habits and medical history, and pre-registered nutritional and health status information of other users obtained by examinations other than the urine test kit, the other users having analysis results similar to those of the user.

### TECHNICAL FIELD

The present invention relates to a nutritional and health status analysis system using a urine test kit.

### BACKGROUND ART

In recent years, with growing health consciousness, an increasing number of people have been consuming health foods and supplements. In order for a user to select health foods and the like that are suitable for the user, it is important to accurately understand the user's nutritional and health status. Based on a body fluid such as blood or urine, it is possible to understand the condition of one's body, that is, the presence or absence of a disease and the nutritional and health status of the body. However, in order for a user to know the user's own nutritional and health status, it has been necessary to use a special and expensive testing apparatus for blood tests and urine tests and to have the tests performed at a hospital or the like, and therefore such tests have not been readily accessible to everyone.

A urine test system using spot urine has been proposed as a system that allows anyone to easily use a urine test (see Patent Literature 1). This urine test system is intended to improve the convenience of urine testing for a user, and uses a system in which a client terminal on a user side and a server on a service provider side are connected via a network to transmit and receive data, such that the server evaluates and determines urine components and outputs result data of the evaluation and determination.

### CITATION LIST

### Patent Literature

### Patent Literature 1: JP 7281856 B2

### SUMMARY OF INVENTION

### Technical Problem

However, with the technology described above, it is necessary to transmit and receive the evaluation results between the server and the client terminal, which is time-consuming, precludes offline determination, entails a risk of information leakage due to transmission of personal information to the server, and complicates evaluation and determination operations.

In view of such circumstances, an object of the present invention is to provide a nutritional and health status analysis system that enables a user to easily and quickly know the user's own nutritional and health status offline.

### Solution to Problem

In order to solve the above problems, the present invention relates to a nutritional and health status analysis system, the system including a client terminal,
wherein the client terminal includes:
urine test kit color data input means for inputting color data of a urine test kit immersed in urine collected by a user;
question data input means for inputting question data regarding a physical condition of the user within one week from a day on which the urine is collected;
urine test kit color data analysis means for analyzing the color data of the urine test kit;
question data analysis means for analyzing the question data; and
nutritional and health status evaluation means for evaluating, by machine learning, a nutritional and health status of the user based on
i) basic information regarding the user including sex and age, and registration information regarding the user including lifestyle habits and medical history, and
ii) pre-registered nutritional and health status information of other users, the pre-registered nutritional and health status information being information obtained by examinations other than an examination using the urine test kit, the other users having analysis results similar to the analysis results of the user obtained by analyzing past question data of the user and urine test kit color data of the user.

It should be noted that the client terminal can further include internal algorithm means having data transmission and reception means for receiving the machine-learned analysis results and data of ii).

### Advantageous Effects of Invention

According to the system of the present invention, the client terminal possessed by the user itself can perform nutritional and health evaluation by machine learning using color information of a urine test kit immersed in urine collected by the user, based on basic information regarding other users including sex and age, pre-registered information regarding other users including lifestyle habits and medical history, and pre-registered nutritional and health status information, after the user performs a test on the urine and identifies components in the urine by color. That is, there is no need to transmit and receive data between the server and the client terminal in order to obtain the evaluation results, and offline analysis of nutritional and health status becomes possible. As a result, it is possible to reduce the risk of information leakage due to transmission of personal information to the server.

In addition, if necessary, the client terminal can further include internal algorithm means having data transmission and reception means for receiving the machine-learned analysis results and data of ii). As a result, it is possible to update and renew the analysis results and data to be used for the machine learning described above, thereby enabling more accurate offline evaluation of nutritional and health status.

### BRIEF DESCRIPTION OF DRAWINGS

FIG. 1 is a functional block diagram of a nutritional and health status analysis system of the present invention.

FIG. 2 is a schematic flow diagram of the nutritional and health status analysis system of the present invention.

FIG. 3 is a schematic configuration diagram of a test kit.

FIG. 4 is a screen display image diagram for question data input.

### DESCRIPTION OF EMBODIMENTS

Hereinafter, an example of an embodiment of the present invention will be described in detail with reference to the drawings.

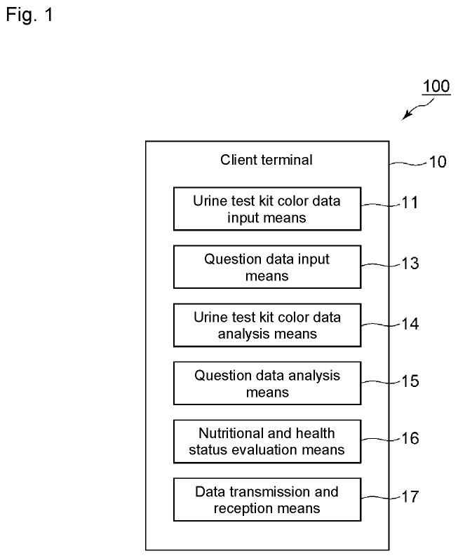

FIG. 1 illustrates a functional block diagram of a nutritional and health status analysis system according to an embodiment. As illustrated in FIG. 1, a nutritional and health status analysis system 100 includes a client terminal 10.

The client terminal 10 includes urine test kit color data input means 11, question data input means 13, urine test kit color data analysis means 14, question data analysis means 15, nutritional and health status evaluation means 16, and data transmission and reception means 17.

The urine test kit color data input means 11 inputs color data of a urine test kit immersed in collected urine, and the question data input means 13 inputs question data regarding a physical condition within one week from a day on which the urine is collected.

The urine test kit color data analysis means 14 analyzes color data of the urine test kit, and the question data analysis means 15 analyzes the question data.

The nutritional and health status evaluation means 16 evaluates a nutritional and health status of the user based on analysis results analyzed by the urine test kit color data analysis means 14 and the question data analysis means 15, respectively, and user information described below. It should be noted that the nutritional and health status evaluation means 16 may include machine learning means and may be configured to learn the analysis results and the user information to evaluate nutritional and health status with higher accuracy.

In addition, the data transmission and reception means 17, for example, receives machine-learned analysis results and data stored in user information storage means of a server (not illustrated) described below from the server (not illustrated), and processes the data by an internal algorithm.

It should be noted that this connection need only be made at the beginning of the operation, and thereafter the nutritional and health status evaluation means 16 can analyze nutritional and health status based on the received information by machine learning. Therefore, the server can be offline, and nutritional and health evaluation can be performed only by the client terminal 10. However, as time passes, the pre-registered information and the nutritional and health status information described above may become outdated, and the analysis accuracy by machine learning of the nutritional and health status evaluation means 16 may decrease. In this case, as appropriate, for example, the client terminal 10 and the server are brought online, and the machine-learned analysis results and data are updated and renewed by internal algorithm means of the client terminal 10 having data transmission and reception means for receiving the machine-learned analysis results and data of ii), thereby making it possible to improve analysis accuracy.

It should be noted that the server includes user information storage means and server-side data transmission and reception means.

The user information storage means stores user information.

User information stored in the user information storage means includes basic information regarding other users including sex and age, pre-registered information regarding other users including lifestyle habits and medical history, and pre-registered nutritional and health status information that is information already obtained for the other users by examinations other than an examination using the urine test kit.

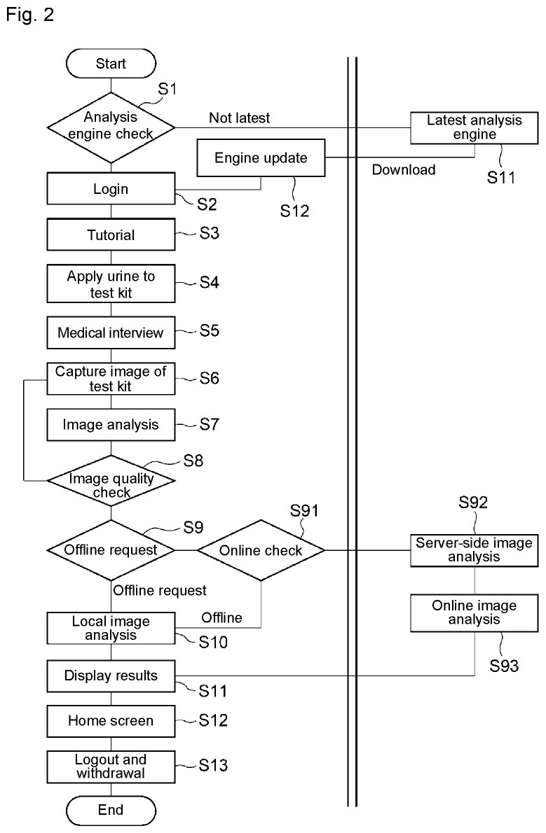

Next, a use flow of the nutritional and health status analysis system according to an embodiment will be described with reference to the drawings. FIG. 2 illustrates a schematic flow diagram of the nutritional and health status analysis system according to the present embodiment.

As illustrated in FIG. 2, first, a user checks whether the analysis engine of the client terminal 10 is the latest (S1). In a case where the analysis engine is the latest analysis engine, the user logs in to the client terminal 10 as it is (S2). In a case where the analysis engine is not the latest, as appropriate, the user accesses a server (not illustrated) (S11), downloads the latest analysis engine (S12), and then logs in to the client terminal 10 (S2).

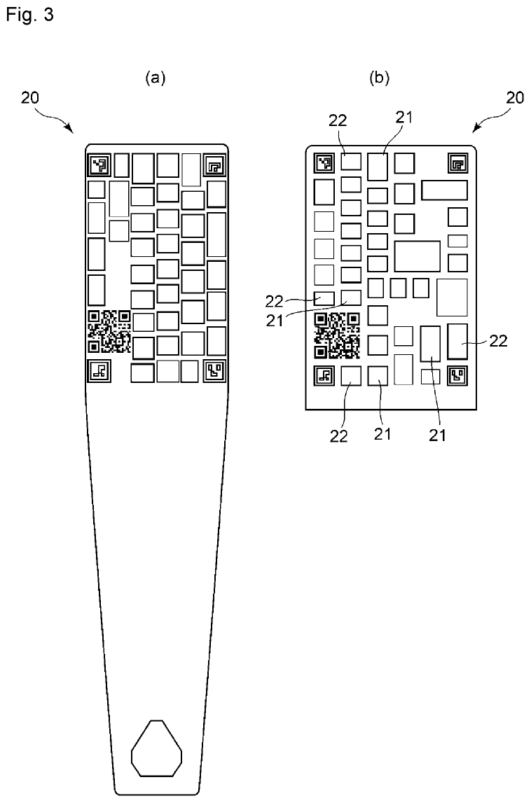

Next, a tutorial is provided to the user to explain how to use the client terminal 10, that is, the nutritional and health status analysis system 100 (S3), and thereafter the user directly applies urine to the test kit and confirms color development of the test kit due to the urine being applied (S4). FIG. 3 illustrates a schematic configuration diagram of a test kit used in the present embodiment. As illustrated in FIG. 3, a test kit 20 of the present embodiment has a plurality of rectangular reaction sites 21 and 22 that develop color by reacting with components in urine, and these reaction sites include color-check sites 21 of four colors and four black sites 22 having brightness different from surrounding brightness. It should be noted that, in order to reduce an influence of light as much as possible, the color-check sites 21 of four colors are disposed separately at positions as far apart as possible on the test kit 20, thereby making it difficult for the color-check sites 21 to simultaneously receive an influence of the same light source, and even if one site has an erroneous brightness due to external light, the influence can be reduced by the other sites 21.

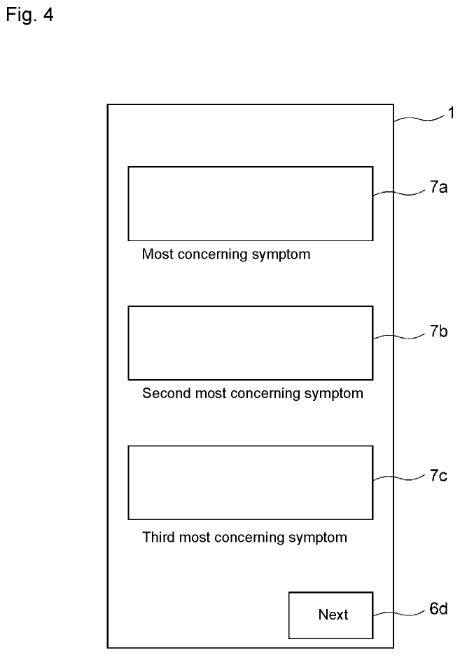

Next, the user answers the questions using the question data input means 13 included in the client terminal 10 (S5). FIG. 4 illustrates a screen display image diagram for question data input. As illustrated in FIG. 4, a display 1 shows symptom selection fields (7a to 7c) and a button 6d. Specifically, the display 1 shows the symptom selection field 7a for inputting a “most concerning symptom”, the symptom selection field 7b for inputting a “second most concerning symptom”, and the symptom selection field 7c for inputting a “third most concerning symptom”. The user selects each of the symptom selection fields (7a to 7c) and then selects, from among displayed items (not shown), an item suitable for the user.

Although only three symptom selection fields are displayed here, four or more symptom selection fields may be displayed, or two or fewer symptom selection fields may be displayed. In addition, the user does not necessarily need to answer all of the provided symptom selection fields, and, for example, may input only the symptom selection field 7a.

It should be noted that the questions are not limited to “symptoms of concern”, and, for example, question items regarding the user's body weight, blood pressure, pulse rate, and the like may be provided. In addition, the input method is not limited to a method of selecting from items prepared in advance, and, for example, a method of answering in free text may be used.

After the user selects an item suitable for the user for each of the symptom selection fields (7a to 7c) and then taps the button 6d labeled “Next”, the client terminal 10 analyzes the question data using the question data analysis means 15.

After input of the question data is completed, the test kit 20 is photographed using a camera (not illustrated) provided in the client terminal 10 (S6), and image analysis is performed (S7). The image analysis is performed based on information on brightness and chromaticity of the four colors 21 for color check and the black color 22 around the pad. At this time, the client terminal 10 confirms whether the image capture is satisfactorily performed. The urine test kit color data input means 11 automatically recaptures an image by the camera means when image-capture data captured by the camera means mounted on the client terminal 10 is not analyzable.

In addition, it is also possible to capture, using a camera, an image of a reaction site of a urine test kit (test kit) that develops color by using collected urine, correct a color image of the reaction site based on a color sample kit, and return the corrected color image to the client terminal. It should be noted that, in this case, when image capture cannot be satisfactorily performed due to a shadow or the like, image quality checking such as discarding the image is performed (S8).

After analyzing the question data and the test kit 20, the client terminal 10 evaluates the nutritional and health status of the user offline by machine learning using the nutritional and health status evaluation means 16 (S9 and S10). Specifically, the nutritional and health status of the user is evaluated by machine learning based on pre-registered nutritional and health status information already obtained for other users by examinations other than an examination using the test kit, the other users having information similar to analysis results of past question data of the user and urine test kit color data. It should be noted that, in this case, the client terminal 10 can, as appropriate, access a server (not illustrated) by the data transmission and reception means 17 for transmitting and receiving machine-learned analysis results and data (S91), receive the analysis results and data from the server, perform image analysis (S92 and S93) by an internal algorithm, and improve analysis accuracy.

The client terminal 10 displays evaluated nutritional and health data on the display 1 (step S11).

After the nutritional and health data is displayed on the display 1, the display 1 of the client terminal 10 transitions to a home screen (S12), and, by logging out, nutritional and health status evaluation on the client terminal 10 is ended (S13).

As described above, using the test kit 20, the user can perform a test on urine collected by the user, identify a mineral content in urine by color, and evaluate the color information using the client terminal 10 itself, thereby making it possible to quickly understand the nutritional and health status of the user. As the client terminal 10, a PC, a smartphone, a tablet terminal, or the like can be used. Accordingly, the user can easily use the nutritional and health status analysis system.

In addition, since the client terminal 10 further includes the question data input means 13 for inputting question data regarding a physical condition within one week from the day on which urine is collected and the question data analysis means 15, it is possible to evaluate nutritional and health status in consideration of changes in physical condition and symptoms recognized by the user on the day on which urine is collected and on days close thereto.

As a result, it is possible to provide the nutritional and health status analysis system 100 that enables a user to easily and quickly know the user's own nutritional and health status.

In view of the above embodiment, the present application also encompasses the following inventions.
[1] A nutritional and health status analysis system, the system including a client terminal,
wherein the client terminal includes:
urine test kit color data input means for inputting color data of a urine test kit immersed in urine collected by a user;
question data input means for inputting question data regarding a physical condition of the user within one week from a day on which the urine is collected;
urine test kit color data analysis means for analyzing the color data of the urine test kit;
question data analysis means for analyzing the question data; and
nutritional and health status evaluation means for evaluating, by machine learning, a nutritional and health status of the user based on
i) basic information regarding the user including sex and age, and registration information regarding the user including lifestyle habits and medical history, and
ii) pre-registered nutritional and health status information of other users, the pre-registered nutritional and health status information being information obtained by examinations other than an examination using the urine test kit, the other users having analysis results similar to the analysis results of the user obtained by analyzing past question data of the user and urine test kit color data of the user.
[2] The nutritional and health status analysis system according to [1], wherein the client terminal further includes internal algorithm means having data transmission and reception means for receiving the machine-learned analysis results and data of ii).
[3] The nutritional and health status analysis system according to [1] or [2], wherein the urine test kit develops color by reacting with components in urine, and has a color intensity that varies depending on concentrations of the components.
[4] The nutritional and health status analysis system according to any one of [1] to [3], wherein the urine test kit color data input means captures, by camera means mounted on the client terminal, an image of a reaction site of the urine test kit that develops color by using collected urine, performs correction of a color image of the reaction site based on a color sample, and returns the corrected color image to the client terminal.
[5] The nutritional and health status analysis system according to any one of [1] to [4], wherein the urine test kit color data input means automatically recaptures an image by the camera means when image-capture data captured by the camera means mounted on the client terminal is not analyzable.
[6] The nutritional and health status analysis system according to any one of [1] to [5], wherein the system does not depend on an online environment.

Although some embodiments of the present invention have been described above, these embodiments are presented by way of example and are not intended to limit the scope of the invention. These novel embodiments can be implemented in various other forms, and various omissions, substitutions, and modifications can be made without departing from the gist of the invention. These embodiments and variations thereof are included in the scope and gist of the invention and are also included in the invention described in the claims and equivalents thereof.

### REFERENCE SIGNS LIST

1 Display
10 Client terminal
11 Urine test kit color data input means
13 Question data input means
14 Urine test kit color data analysis means
15 Question data analysis means
16 Nutritional and health status evaluation means
17 Data transmission and reception means
20 Test kit
21, 22 Reaction site
100 Nutritional and health status analysis system

1. A nutritional and health status analysis system, the system comprising a client terminal,
wherein the client terminal includes:
a urine test kit color data input unit for inputting color data of a urine test kit immersed in urine collected by a user;
a question data input unit for inputting question data regarding a physical condition of the user within one week from a day on which the urine is collected;
a urine test kit color data analysis unit for analyzing the color data of the urine test kit;
a question data analysis unit for analyzing the question data; and
a nutritional and health status evaluation unit for evaluating, by machine learning, a nutritional and health status of the user based on
i) basic information regarding the user including sex and age, and registration information regarding the user including lifestyle habits and medical history, and
ii) pre-registered nutritional and health status information of other users, the pre-registered nutritional and health status information being information obtained by examinations other than an examination using the urine test kit, the other users having analysis results similar to the analysis results of the user obtained by analyzing past question data of the user and urine test kit color data of the user.

2. The nutritional and health status analysis system according to claim 1, wherein the client terminal further includes an internal algorithm unit having data transmission and a reception unit for receiving the machine-learned analysis results and data of ii).

3. The nutritional and health status analysis system according to claim 1, wherein the urine test kit develops color by reacting with components in urine, and has a color intensity that varies depending on concentrations of the components.

4. The nutritional and health status analysis system according to claim 1, wherein the urine test kit color data input unit captures, by a camera mounted on the client terminal, an image of a reaction site of the urine test kit that develops color by using collected urine, performs correction of a color image of the reaction site based on a color sample, and returns the corrected color image to the client terminal.

5. The nutritional and health status analysis system according to claim 4, wherein the urine test kit color data input unit automatically recaptures an image by the camera when image-capture data captured by the camera mounted on the client terminal is not analyzable.

6. The nutritional and health status analysis system according to claim 1, wherein the system does not depend on an online environment.
